(19) United States
(12) Patent Application Publication
(10) Pub. No.: US 2022/0125540 A1
(43) Pub. Date: Apr. 28, 2022

(54) ROBOTIC SURGICAL INSTRUMENT INCLUDING HIGH ARTICULATION WRIST ASSEMBLY

(71) Applicant: Covidien LP, Mansfield, MA (US)

(72) Inventor: Zachary Traina, Verona, NJ (US)

(21) Appl. No.: 17/427,980

(22) PCT Filed: Feb. 12, 2020
(86) PCT No.: PCT/US20/17891

§ 371 Date: Aug. 3, 2021

Related U.S. Application Data
(60) Provisional application No. 62/806,443, filed on Feb. 15, 2019.

(51) Int. Cl. A61B 34/00; A61B 34/37; A61B 18/14

(52) U.S. Cl. CPC A61B 34/71; A61B 2034/305; A61B 18/1445; A61B 34/37

(57) ABSTRACT

A robotic electromechanical surgical instrument includes a housing, an elongated shaft extending distally from the housing, a wrist assembly supported on the elongated shaft, an end effector coupled to the wrist assembly, and first, second, and third cables coupled to the wrist assembly. The elongated shaft defines a longitudinal axis and the wrist assembly articulates relative to the longitudinal axis. The wrist assembly includes a first interface, a first link pivotally coupled to the first interface, a second link coupled to the first link, and a third link pivotally coupled to the second link. The first cable is coupled to the second link such that proximal axial translation thereof causes the second link to pivot about a first pivot axis. The third cable is coupled to the third link such that axial translation thereof causes the third link to pivot about a second pivot axis.

### BACKGROUND

Robotic surgical systems have been used in minimally invasive medical procedures. Some robotic surgical systems include a console supporting a surgical robotic arm and a surgical instrument having at least one end effector (e.g., a forceps or a stapling device) mounted to the robotic arm. The robotic arm provides mechanical power to the surgical instrument for its operation and movement. Each robotic arm may include an instrument drive unit that is operatively connected to the surgical instrument. The surgical instruments may include cables that are motor driven to operate end effectors of the surgical instruments.

### SUMMARY

The present disclosure relates to surgical instruments for use in surgical procedures. More specifically, the present disclosure relates to articulable robotic surgical instruments for robotic surgical systems used to conduct minimally invasive surgical procedures. The present disclosure provides for reduced size surgical instruments for robotic surgical systems that provide increased articulation, torque transmission, and mechanical manipulation.

In accordance with an aspect of the present disclosure, a robotic electromechanical surgical instrument is provided. The robotic electromechanical surgical instrument includes a housing, an elongated shaft extending distally from the housing, a wrist assembly supported on the elongated shaft, an end effector coupled to the wrist assembly, and first, second, and third cables coupled to the wrist assembly. The elongated shaft defines a longitudinal axis and the wrist assembly is configured to articulate relative to the longitudinal axis. The wrist assembly includes a first interface, a first link pivotally coupled to the first interface, a second link coupled to the first link, and a third link pivotally coupled to the second link.

The first cable is coupled to the second link such that proximal axial translation of the first cable along the longitudinal axis causes the second link to pivot about a first pivot axis in a first direction. The second cable is coupled to the second link such that proximal axial translation of the second cable along the longitudinal axis causes the second link to pivot about the first pivot axis in a second direction opposite the first direction. The third cable is coupled to the third link such that axial translation of the third cable along the longitudinal axis causes the third link to pivot about a second pivot axis. In an aspect, the first link and the second link are coupled together such that proximal axial translation of the first cable along the longitudinal axis causes the second link and the first link to pivot together about the first pivot axis.

The third cable may be coupled to the third link such that proximal axial translation along the longitudinal axis of a first portion of the third cable and simultaneous distal axial translation along the longitudinal axis of a second portion of the third cable causes the third link to pivot about the second pivot axis in a third direction. Additionally, or alternatively, the third cable is coupled to the third link such that distal axial translation along the longitudinal axis of the first portion of the third cable and simultaneous proximal axial translation along the longitudinal axis of the second portion of the third cable causes the third link to pivot about the second pivot axis in a fourth direction opposite the third direction.

In an aspect, the robotic electromechanical surgical instrument includes an electrical cable operably coupled to a portion of at least one of the end effector or the wrist assembly. The electrical cable may configured to transmit a sensor signal from at least one of the end effector or the wrist assembly. Additionally, or alternatively, the electrical cable is configured to transmit electrosurgical treatment energy to a portion of the end effector. The housing may include an electrical contact disposed thereon and the electrical cable is coupled to the electrical contact.

In an aspect, a firing assembly is operably coupled to the end effector and configured to control an operation of the end effector.

In an aspect, the first interface includes a first half and a second half. The first half defines a first cable channel slidably supporting the first cable therein and a third cable channel slidably supporting a first portion of the third cable therein. Additionally, or alternatively, the second half defines a second cable channel slidably supporting the second cable therein and a fourth cable channel slidably supporting a second portion of the third cable therein. The second half of the first interface may further define an electrical cable channel configured to slidably support an electrical cable therein.

In accordance with another aspect of the present disclosure, a wrist assembly for use with an electromechanical surgical instrument is provided. The wrist assembly includes a first interface defining a longitudinal axis, a first link pivotally coupled to the first interface and configured to pivot relative to the first interface about a first pivot axis, a second link coupled to the first link and axially aligned with the first link, and a third link pivotally coupled to the second link and configured to pivot relative to the second link about a second pivot axis. Additionally, the wrist assembly includes first, second, and third cables. The first cable is coupled to the second link such that proximal axial translation of the first cable along the longitudinal axis causes the second link to pivot about the first pivot axis in a first direction. The second cable is coupled to the second link such that proximal axial translation of the second cable along the longitudinal axis causes the second link to pivot about the first pivot axis in a second direction opposite the first direction. The third cable is coupled to the third link such that axial translation of the third cable along the longitudinal axis causes the third link to pivot about the second pivot axis.

In an aspect, the first link and the second link are coupled together such that proximal axial translation of the first cable along the longitudinal axis causes the second link and the first link to pivot together about the first pivot axis. The third cable may be coupled to the third link such that proximal axial translation along the longitudinal axis of a first portion of the third cable and simultaneous distal axial translation along the longitudinal axis of a second portion of the third cable causes the third link to pivot about the second pivot axis in a third direction. Additionally, or alternatively, the third cable is coupled to the third link such that distal axial translation along the longitudinal axis of the first portion of the third cable and simultaneous proximal axial translation along the longitudinal axis of the second portion of the third cable causes the third link to pivot about the second pivot axis in a fourth direction opposite the third direction.

In an aspect, the wrist assembly further includes an electrical cable operably coupled to a portion of the wrist assembly. The electrical cable may be configured to transmit a sensor signal from the wrist assembly.

In an aspect, the first interface includes a first half and a second half. The first half may define a first cable channel slidably supporting the first cable therein and a third cable channel slidably supporting a first portion of the third cable therein. Additionally, or alternatively, the second half defines a second cable channel slidably supporting the second cable therein and a fourth cable channel slidably supporting a second portion of the third cable therein. The second half of the first interface further may define an electrical cable channel configured to slidably support an electrical cable therein. In an aspect, the first interface defines a central channel along the longitudinal axis configured to support a portion of a drive assembly therethrough.

Other aspects, features, and advantages provided by some or all of the illustrative embodiments described herein will be apparent from the description, the drawings, and the claims that follow.

### BRIEF DESCRIPTION OF THE DRAWINGS

The accompanying drawings, which are incorporated in and constitute a part of this specification, illustrate embodiments of the present surgical instruments for robotic surgical systems and, together with a general description of the disclosure given above, and the detailed description of the embodiment(s) given below, serve to explain the principles of the disclosure, wherein:

FIG. 1 is a schematic illustration of a robotic surgical system in accordance with the present disclosure;

FIG. 2A is a perspective view of a surgical instrument of the robotic surgical system of FIG. 1 in an unarticulated position;

FIG. 2B is a rear perspective view of a proximal portion of a surgical instrument of the robotic surgical system of FIG. 1;

FIG. 3 is an enlarged, perspective view of the indicated area of detail shown in FIG. 2A;

FIG. 4 is a perspective view of a distal portion of an elongated shaft and a wrist assembly, with parts separated, of a surgical instrument of the robotic surgical system of FIG. 1;

FIG. 5 is a perspective view of the wrist assembly of FIG. 4 with portions thereof shown in phantom for clarity;

FIG. 6 is an enlarged, cross-sectional view of the wrist assembly of FIG. 5 as taken along section line 6-6 of FIG. 5;

FIG. 7 is an enlarged, cross-sectional view of the wrist assembly of FIG. 5 as taken along section line 7-7 of FIG. 6;

FIG. 8 is a perspective view of a surgical instrument of the robotic surgical system of FIG. 1 in one articulated position;

FIG. 9 is an enlarged view of the indicated area of detail shown in FIG. 8;

FIG. 10 is a perspective view of a surgical instrument of the robotic surgical system of FIG. 1 in another articulated position;

FIG. 11A is a perspective view of an end effector of an aspect of the robotic surgical system of FIG. 1 having an energy delivery device; and

FIG. 11B is a perspective view of an end effector of an aspect of the robotic surgical system of FIG. 1 having an energy delivery device.

### DETAILED DESCRIPTION

Embodiments of the present surgical instruments for robotic surgical systems are described in detail with reference to the drawings, in which like reference numerals designate identical or corresponding elements in each of the several views. As used herein, the term “distal” refers to structure that is closer to a patient, while the term “proximal” refers to structure farther from the patient.

As used herein, the term “clinician” refers to a doctor, nurse, or other care provider and may include support personnel. In the following description, well-known functions or constructions are not described in detail to avoid obscuring the present disclosure in unnecessary detail.

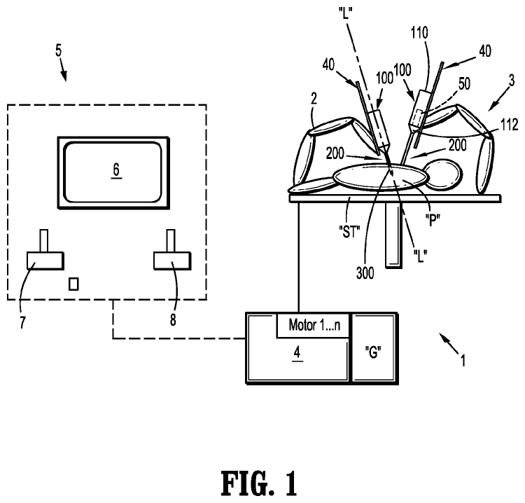

Referring initially to FIG. 1, a surgical system, such as, for example, a robotic surgical system 1, generally includes one or more surgical robotic arms 2, 3, a control device 4, and an operating console 5 coupled with control device 4. Any of the surgical robotic arms 2, 3 may have a robotic surgical assembly 100 and an electromechanical surgical instrument 200 coupled thereto. Electromechanical surgical instrument 200 includes an end effector 300 disposed at a distal portion thereof. In some embodiments, robotic surgical assembly 100 may be removably attached to a slide rail 40 of one or more of surgical robotic arms 2, 3. In certain embodiments, robotic surgical assembly 100 may be fixedly attached to slide rail 40 of one or more of surgical robotic arms 2, 3.

Operating console 5 of robotic surgical system 1 includes a display device 6, which is set up to display three-dimensional images, and manual input devices 7, 8, by means of which a clinician (not shown) is able to telemanipulate the robotic arms 2, 3 of robotic surgical system 1 in a first operating mode, as known in principle to a person skilled in the art. Each robotic arm of robotic arms 2, 3 may be composed of any number of members, which may be connected through any number of joints. Robotic arms 2, 3 may be driven by electric drives (not shown) that are connected to control device 4. Control device 4 (e.g., a computer) of robotic surgical system 1 is set up to activate the drives, for example, by means of a computer program, in such a way that robotic arms 2, 3, the attached robotic surgical assembly 100, and thus electromechanical surgical instrument 200 (including end effector 300) of robotic surgical system 1 execute a desired movement according to a movement defined by means of manual input devices 7, 8. Control device 4 may be set up in such a way that it regulates movement of robotic arms 2, 3 and/or of the drives.

Robotic surgical system 1 is configured for use on a patient “P” positioned (e.g., lying) on a surgical table “ST” to be treated in a minimally invasive manner by means of a surgical instrument, e.g., electromechanical surgical instrument 200 and, more specifically, end effector 300 of electromechanical surgical instrument 200. Robotic surgical system 1 may include more than two robotic arms 2, 3, and the additional robotic arms are likewise connected to control device 4 and telemanipulatable by means of operating console 5. A surgical instrument, for example, electromechanical surgical instrument 200 (including end effector 300 thereof), may also be attached to any additional robotic arm(s).

Control device 4 of robotic surgical system 1 may control one or more motors (not shown), each motor configured to drive movement of robotic arms 2, 3 in any number of directions. Control device 4 may control an instrument drive unit 110 including one or more motors 50 (or motor packs). Motors 50 drive various operations of end effector 300 of electromechanical surgical instrument 200. Motors 50 may include a rotation motor, such as, for example, a canister motor. One or more of motors 50 (or a different motor, not shown) may be configured to drive a rotation of electromechanical surgical instrument 200, or components thereof, relative to a longitudinal axis “L-L” thereof. The one or more motors can be configured to effect operation and/or movement of electromechanical end effector 300 of electromechanical surgical instrument 200.

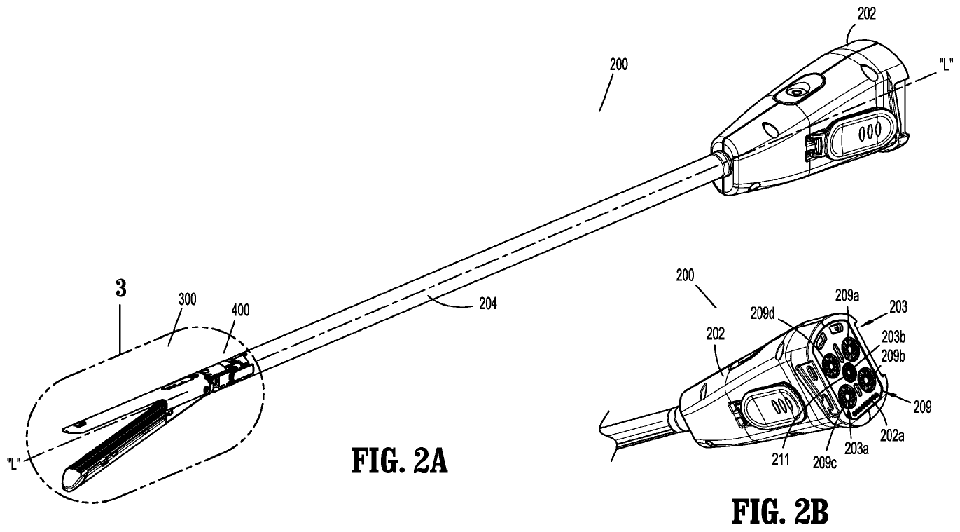

Turning now to FIGS. 2A-2B, electromechanical surgical instrument 200 of robotic surgical system 1 includes a housing 202 at a proximal end portion thereof and an elongated shaft 204 that extends distally from housing 202. Elongated shaft 204 includes a wrist assembly 400 supported on a distal end portion of elongated shaft 204 that couples end effector 300 to elongated shaft 204. Briefly, and as will be described in greater detail below, wrist assembly 400 includes, among additional components, a first interface 401, a first link 405 pivotally coupled to the first interface 401, a second link 409 coupled to the first link 405, and a third link 411 pivotally coupled to the second link 409.

Housing 202 of electromechanical surgical instrument 200 is configured to selectively couple to instrument drive unit 110 of robotic surgical assembly 100, for example, via side loading on a sterile interface module 112 of robotic surgical assembly 100, to enable motors 50 of instrument drive unit 110 of robotic surgical assembly 100 to operate end effector 300 of electromechanical surgical instrument 200. Housing 202 of electromechanical surgical instrument 200 supports a drive assembly 203 that mechanically and/or electrically cooperates with motors 50 of instrument drive unit 110 of robotic surgical assembly 100.

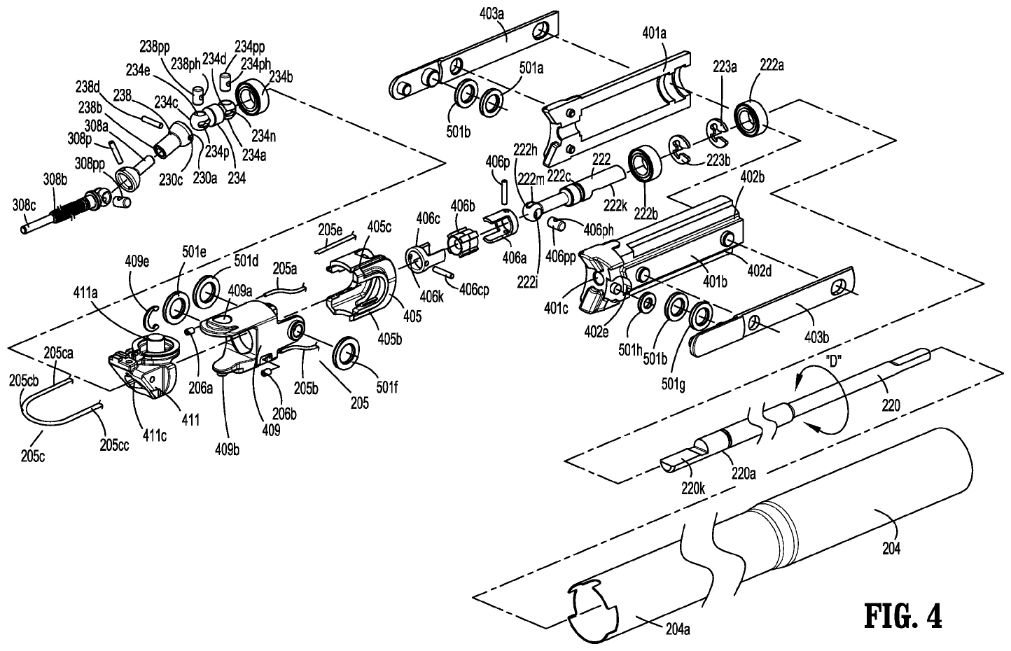

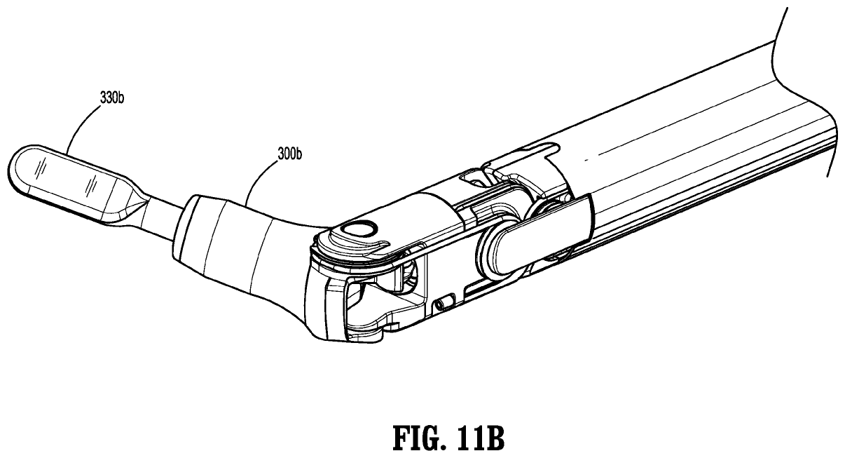

Additionally, housing 202 includes an electrical contact 202e (FIG. 2B) on a proximal portion thereof which interfaces with a corresponding electrical contact (not shown) of instrument drive unit 110 to create an electrical connection between electrical cable 205e (FIG. 4) and the other components of robotic surgical system 1 (e.g., an electrosurgical generator, controller, sensor, etc.). It is contemplated that electromechanical surgical instrument 200 may additionally include a printed circuit board (not shown) to which electrical cable 205e is coupled. Electrical cable 205e may be utilized to create an electrical connection between any portion of electromechanical surgical instrument 200 (e.g., end effector 300) and any component(s) of robotic surgical system 1 (e.g., robotic arms 2, 3, control device 4, and/or operating console 5). In one aspect, electrical cable 205e is used to transmit electrosurgical treatment energy from an electrosurgical generator “G” (FIG. 1) to a portion of end effector 300, such as an energy delivery portion or device (FIGS. 11A-11B) coupled to end effector 300. For example, any portion of end effector may be configured to transmit electrosurgical energy generated from generator “G” to the surgical site (e.g., tissue, vessel, lesion, etc.). Additionally, or alternatively, with brief reference to FIG. 11A, end effector 300a may include a bipolar forceps with one or more seal plates 320a attached to jaw members 310a thereof, where the one or more seal plates 320a transmit electrosurgical energy generated by generator “G” to the surgical site (e.g., tissue, vessel, lesion, etc.). Additionally, or alternatively, end effector 300b may include an energy delivery element 330b shown in FIG. 11B coupled thereto. Any components of the above described embodiments are contemplated as being usable with other embodiments even if not explicitly described or shown.

Additionally, or alternatively, electrical cable 205e may be utilized to transmit sensor signals between end effector 300 (or sensors coupled thereto) and any other component(s) of robotic surgical system 1. Although only a single electrical cable is shown and described, it is contemplated that multiple electrical cables may be utilized, or alternatively, that electrical cable 205e may include multiple independent electrical cables therein.

Drive assembly 203 of electromechanical surgical instrument 200 can include any suitable electrical and/or mechanical component to effectuate driving force/movement, and which components may be similar to components of the drive assembly described in commonly owned International Application Publication No. WO2017053358, filed Sep. 21, 2016, the entire disclosure of which is incorporated by reference herein. In particular, as seen in FIG. 2B, drive assembly 203 of electromechanical surgical instrument 200 includes a cable drive assembly 203a and a firing assembly 203b. The cable drive assembly 203a is similar to that described in commonly owned U.S. Patent Application Publication No. 2015/0297199, filed Oct. 22, 2015 and entitled “Adapter Assembly with Gimbal for Interconnecting Electromechanical Surgical Devices and Surgical Loading Units, and Surgical Systems Thereof,” the entire disclosure of which is incorporated by reference herein.

With reference to FIG. 2B, cable drive assembly 203a of electromechanical surgical instrument 200 includes one or more driven members 209, such as first driven member 209a, second driven member 209b, third driven member 209c, and fourth driven member 209d to enable robotic surgical assembly 100 to transfer power and actuation forces from motors 50 of robotic surgical assembly 100 to ultimately drive movement of components of end effector 300 of electromechanical surgical instrument 200.

Cable drive assembly 203a of electromechanical surgical instrument 200 includes cables 205 (FIG. 4), such as first cable 205a, second cable 205b, and third cable 205c. First cable 205a and second cable 205b are coupled to a respective driven member 209a, 209b, (FIG. 2B) of electromechanical surgical instrument 200 at a proximal end portion thereof. First cable 205a and second cable 205b of cable drive assembly 203a extend distally to distal end portions thereof, and may include ferrules 206a, 206b (FIG. 4), respectively, that couple to a component of wrist assembly 400 (e.g., second link 409). Additionally, one end of third cable 205c may be coupled to driven member 209c while the other end of third cable 205 is coupled to driven member 209d. A mid portion of third cable 205c wraps around a component of wrist assembly 400 (e.g., third link 411).

Cables 205, when controlled by driven members 209, effectuate an articulation//pitch/yaw of wrist assembly 400 of electromechanical surgical instrument 200 and end effector 300 of electromechanical surgical instrument 200 upon actuation of one or more of cables 205. Cable drive assembly 203a can include one or more pulleys, friction wheels, gears, couplers, rack and pinion arrangements, etc. coupled directly or indirectly to driven members 209 and/or cables 205 to facilitate driving movement imparted through driven members 209 and/or cables 205. In one aspect, rotation of any driven member 209 causes longitudinal (axial) translation of a respective cable 205 or cables. A detailed description of the relationship between driven members 209 and cables 205 may be found in U.S. Provisional Application Ser. No. 62/546,066, filed on Aug. 16, 2017, filed as International PCT Application No. PCT/US18/46619, filed on Aug. 14, 2018, the entire contents of which are incorporated by reference herein. The cables 205 can be arranged such that diagonal cables can be positioned to be driven in opposite directions in order to provide articulation in multiple axes (e.g., two). Although only three cables are shown, cable drive assembly 203a can include any number of cables, for example, to provide additional functionally at the end effector 300.

As described above, a proximal portion of electrical cable 205e is coupled to an electrical contact 202e (FIG. 2B) on a proximal portion of housing 202 which interfaces with a corresponding electrical contact (not shown) of instrument drive unit 110 to create an electrical connection between electrical cable 205e (FIG. 4) and the other components of robotic surgical system 1 (e.g., an electrosurgical generator, controller, sensor, etc.). Distal of the electrical contact 202e, electrical cable 205e is disposed along electrical cable channel 402e defined in second half 401b of first interface 401, wraps around electrical cable pulley 501h, is disposed along electrical cable channel 405e of first link 405, and may additionally pass through second link 409 and third link 411 to couple to a portion of end effector 300. A relief mechanism (not shown) may also be coupled to electrical cable 205e to relieve any stretching and compensate for any slack that may be effected upon electrical cable 205e upon articulation of articulation assembly 400. Electrical cable pulley 501h is rotatably secured to second half 401b of first interface 401 via securement member 403b.

As described above, a proximal portion of first cable 205a is coupled to driven member 209a such that rotation of driven member 209a effects axial translation of first cable 205a. Distal of the driven member 209a, first cable 205a is slidably disposed along cable channel 402a defined in first half 401a of first interface 401, wraps around inner pulley 501a, is slidably disposed along cable channel 405a of first link 405, and is secured to second link 409 (for example, via ferrule 206a). Inner pulley 501a is rotatably secured to first half 401a of first interface 401 and first link 405 via securement member 403a.

Additionally, as described above, a proximal portion of second cable 205b is coupled to driven member 209b such that rotation of driven member 209b effects axial translation of second cable 205b. Distal of the driven member 209b, second cable 205b is slidably disposed along cable channel 402b defined in second half 401b of first interface 401, wraps around inner pulley 501b, is slidably disposed along cable channel 405b of first link 405, and is secured to second link 409 (for example, via ferrule 206b). Inner pulley 501b is rotatably secured to second half 401b of first interface 401 and first link 405 via securement member 403b.

Additionally, as described above, a proximal portion of a first end of third cable 205c is coupled to driven member 209c such that rotation of driven member 209c effects axial translation of a first portion 205ca of third cable 205c, and a proximal portion of a second end of third cable 205c is coupled to driven member 209d such that rotation of driven member 209d effects axial translation of a second portion 205cc of third cable 205c. As described in greater detail below, rotation of driven members 209c, 209d in opposite directions effects proximal axial translation of one side of third cable 205c (e.g., portion first portion 205ca) while simultaneously effecting distal axial translation of the other side (e.g., second portion 205cc) of third cable 205c. Driven members 209c, 209d are synchronized such that rotation of driven member 209c in one direction causes equal rotation of driven member 209d in an opposite direction, and vice versa. In this manner, axial translation of a first portion 205ca of third cable 205c is always met with opposite axial translation of a second portion 205cd of third cable 205c at an equal rate, and vice versa.

Distal of the driven member 209c, a first portion 205ca of third cable 205c is slidably disposed along cable channel 402c defined in first half 401a of first interface 401, wraps around outer pulley 501c, wraps around pulley 501d which is coupled to second link 209, and wraps around pulley 501e which is coupled to second link 409. A mid-portion 205cb of third cable 205e wraps around cable channel 411e defined in third link 411. A second portion 205cc of third cable 205e wraps around pulley 501f, wraps around outer pulley 501g, and is slidably disposed along cable channel 402d defined in second half 401b of first interface 401 to couple to driven member 209d. Outer pulley 501c is rotatably secured to first half 401a of first interface 401 and first link 405 via securement member 403a, pulley 501d is secured to second link 409 via securement member 403a, pulley 501e is secured to second link 409 via clip 409e, pulley 501f is secured to second link 409 via securement member 403b, and outer pulley 501g is secured to second half 401b of first interface 401 and first link 405 via securement member 403b.

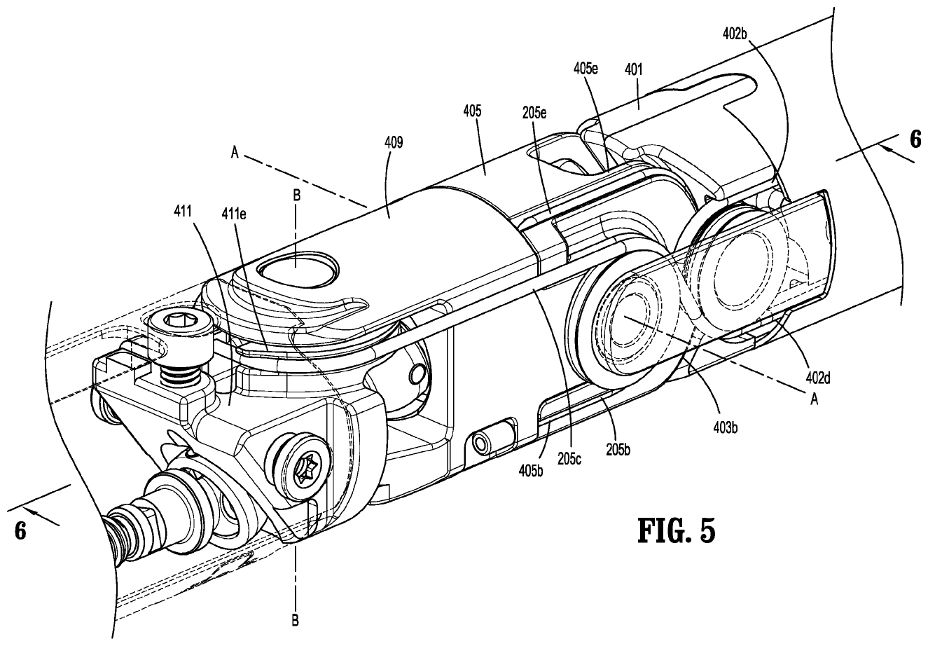

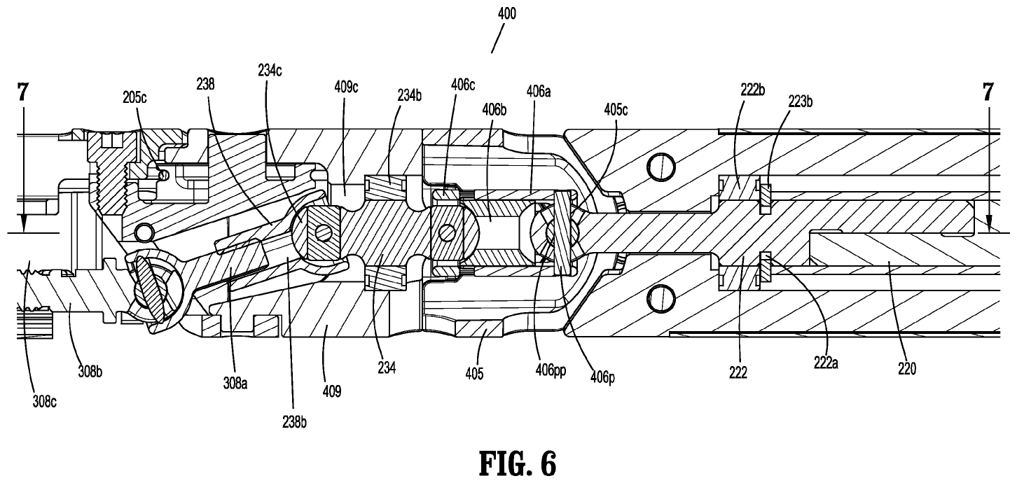

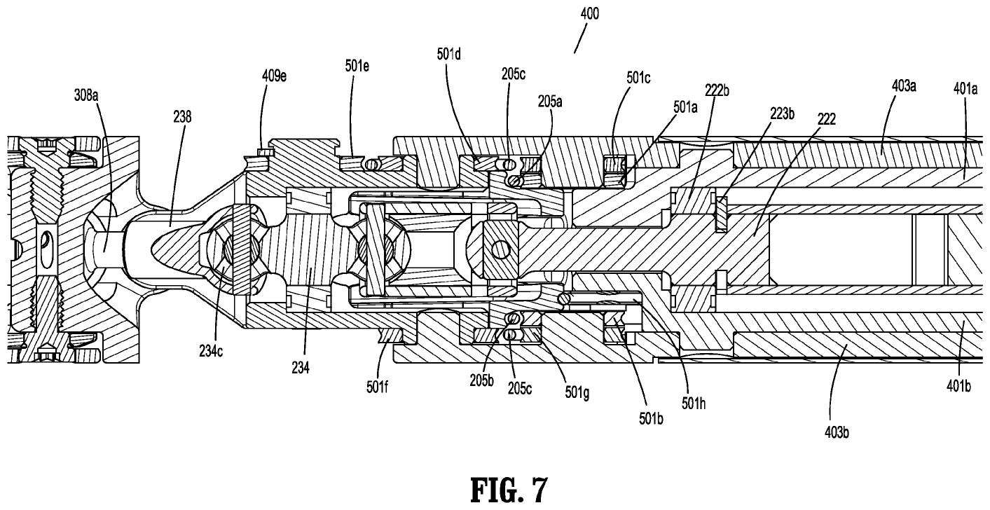

Turning to FIGS. 5 and 6, the components of wrist assembly 400 and drive assembly 203 will now be described. Wrist assembly 400 of elongated shaft 204 of electromechanical surgical instrument 200 includes, from proximal to distal, a first interface 401 coupled to a distal portion of an outer tube 204a of elongated shaft 204, a first link 405 coupled to a distal portion of first interface 401, a second link 409 coupled to a distal portion of first link 405, and a third link 411 coupled to a distal portion of second link 409. First interface 401 defines a longitudinal axis which is aligned with longitudinal axis “L-L” (FIG. 2A) defined by elongated shaft 204. First link 405 is pivotally coupled to first interface 401 via securement members 403a, 403b and may pivot relative to first interface about first pivot axis “A-A”. Second link 409 is axially aligned with first link 405. Third link 411 is pivotally coupled to second link 409 such that third link 411 may pivot relative to second link 409 about second pivot axis “B-B”.

First interface 401 of wrist assembly 400 is formed by first half 401a and second half 401b and defines central aperture 401c that defines a central channel therethrough to receive firing assembly 203b of drive assembly 203. First interface 401 defines cable channels 402a, 402b disposed at circumferentially spaced apart locations thereof to support cables 205a, 205b, respectively. Additionally, first interface 401 defines cable channels 402c, 402d disposed at circumferentially spaced apart locations thereof to support respective portions of third cable 205c therein. Finally, first interface 401 defines electrical cable channel 402e to support electrical cable 205e therein.

First link 405 is pivotally coupled to first interface 401 via securement members 403a, 403b such that first link 405 may pivot relative to first interface 401 via rotation about first pivot axis “A-A”. First link 405 defines a central aperture 405c through which a distal portion of ball shaft 222, a proximal ball housing 406a, an intermediate housing 406b, and a distal ball housing 406c are disposed.

Second link 409 is coupled to first link 405 and secured thereto by compression of any of cables 205, welding, interface fit, or any other suitable means. Second link 409 defines a central aperture 409c, through which a dual ball shaft 234 and a drive coupler 238 are disposed. Dual ball shaft 234 is rotatably coupled to the second link 409 via bearing 234b.

Third link 411 is pivotally coupled to second link 409 such that third link 411 may pivot relative to second link 409 about second pivot axis “B-B”. Second link 409 defines an aperture 409a on a top portion thereof which receives a protrusion 411a defined by third link 411 to secure third link 411 to second link 409. Additionally, second link 409 defines a protrusion 409b on a bottom portion thereof which mates with an aperture (not shown) defined by third link 411. Aperture 409a and protrusion 409b of second link 409 are axially aligned and define second pivot axis “B-B.”

Third link 411 defines a central aperture 411c through which drive coupler 238 and drive coupler 308a are disposed.

Turning now to the components of firing assembly 203b of electromechanical surgical instrument 200, which is in the form of a multi-stage universal joint assembly, firing assembly 203b of drive assembly 203 includes a drive shaft 220 and a ball shaft 222 that extends distally from drive shaft 220. A first bearing 222a is supported on drive shaft 220 to rotatably support drive shaft 220 at a proximal portion of first interface 401 within central aperture 401c. A second bearing 222b is supported on ball shaft 222 to rotatably support ball shaft 222 at a distal portion of first interface 401 within central aperture 403c.

Drive shaft 220 of firing assembly 203b of drive assembly 203 has a proximal end portion coupled to a driven member 211 (FIG. 2B) of drive assembly 203 that operably couples to one or more of motors 50 of robotic surgical assembly 100 (FIG. 1) to enable drive shaft 220 to rotate about longitudinal axis “L-L,” as indicated by arrows “D” (FIG. 4). Drive shaft 220 extends to a keyed distal portion 220k configured to be received by a proximal portion of ball shaft 222. Keyed distal portion 220k is shown with a D-shaped configuration, but may have any suitable non-circular configuration such as a triangle, square, rectangle, star, etc. Drive shaft 220 defines an annular clip channel 220a in an outer surface thereof. Annular clip channel 220a is configured to receive a clip 223a (e.g., an E-clip) to obstruct axial movement of first bearing 222a to enable first bearing 222a of firing assembly 203b to be maintained axially fixed on a surface of drive shaft 220.

A proximal portion of ball shaft 222 of firing assembly 203b defines a keyed portion 222k (FIG. 4) therein, that is configured to mate with keyed distal portion 220k of drive shaft 220, to enable ball shaft 222 to rotate with drive shaft 220. Keyed portion 222k can have any suitable non-circular configuration and may be configured to complement keyed distal portion 220k of drive shaft 220 to facilitate a rotatably locked connection between ball shaft 222 and drive shaft 220 such that ball shaft 222 and drive shaft 220 rotate together. Ball shaft 222 defines an annular clip channel 222c in an outer surface thereof. Annular clip channel 222c is configured to receive a clip 223b (e.g., an E-clip) to obstruct axial movement of bearing 222b to enable bearing 222b of firing assembly 203b to be maintained axially fixed on a surface of ball shaft 222.

Ball shaft 222 further includes a ball member 222h supported on a distal end portion of ball shaft 222. Ball member 222h of ball shaft 222 defines a transverse opening 222i therethrough configured to receive a ball pin 406pp defining a pin hole 406ph therein. Ball member 222h further defines an elongated slot 222m that is configured to align with pin hole 406ph of ball pin 406pp.

Ball shaft 222 is coupled to proximal ball housing 406a via pin 406p and pin 406pp. Proximal ball housing 406a is coupled to distal ball housing 406c while an intermediate portion 406b is disposed between proximal ball housing 406a and distal ball housing 406c. A proximal portion of dual ball shaft 234 is coupled to distal ball housing 406c via pin 406cp and pin 234pp. In particular, distal ball housing 406c defines a pin passage 406k that receives pin 406cp therein to rotatably/articulatably couple the dual ball shaft 234 to the distal ball housing 406c. A distal portion of dual ball shaft 234 is coupled to drive coupler 238 via pin 238d and pin 238pp.

Dual ball shaft 234 of firing assembly 203b includes a proximal ball member 234a that extends proximally from a bearing support surface, and a distal ball member 234c that extends distally from the bearing support surface that rotatably supports third bearing 234b. Proximal and distal ball members 234a, 234c define transverse openings 234d, 234e therethrough, respectively, and elongated slots 234n, 234p therethrough, respectively. Transverse openings 234d, 234e of proximal and distal ball members 234a, 234c are configured to receive ball pins 234pp, 238pp therein, respectively. Each ball pin 234pp, 238pp defines a pin hole 234ph, 238ph, respectively, therein. Pin hole 234ph of ball pin 234pp and elongated slot 234n of ball member 234a are configured to receive pin 406cp of distal ball housing 406 to rotatably/articulatably couple dual ball shaft 234 to distal ball housing 406c (e.g., to define universal joints).

Drive coupler 238 of firing assembly 203b defines a proximal bore 238a (FIG. 4) that rotatably receives distal ball member 234c of second dual ball shaft 234, and a distal bore 238b that is configured to couple to end effector 300 of electromechanical surgical instrument 200. Although distal bore 238b of drive coupler 238 is shown including a non-circular transverse cross-sectional profile or configuration, such as a D-shaped configuration, distal bore 238b can have any non-circular configuration (e.g., triangular, rectangular, pentagonal, etc.) to facilitate a rotatably locked connection between firing assembly 203b and end effector 300 so that end effector 300, or components thereof, can rotate with firing assembly 203b of drive assembly 203. Drive coupler 238 further defines a pin hole 238c that receives a pin 238d to rotatably couple drive coupler 238 to distal ball member 234c of dual ball shaft 234.

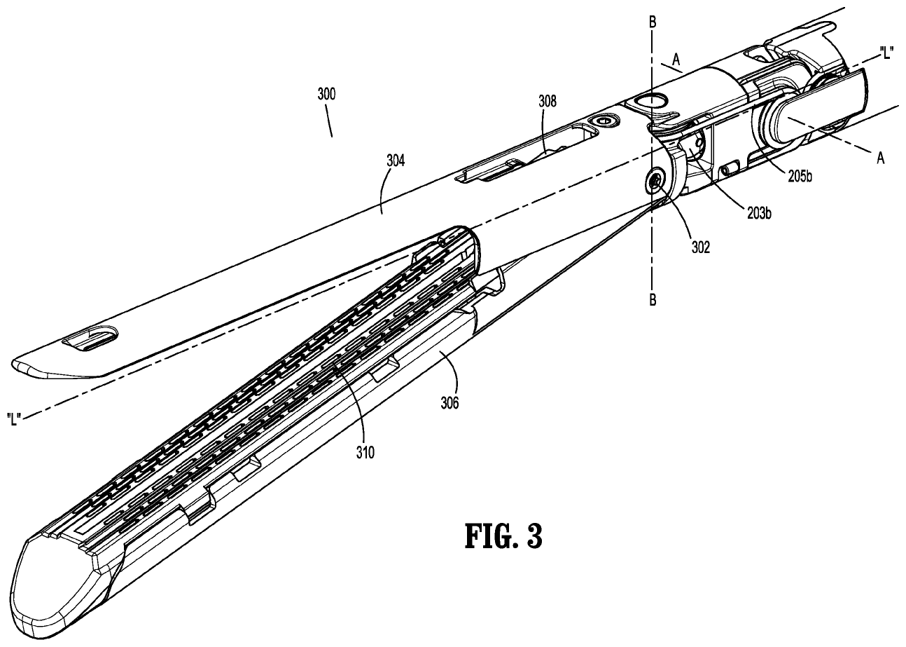

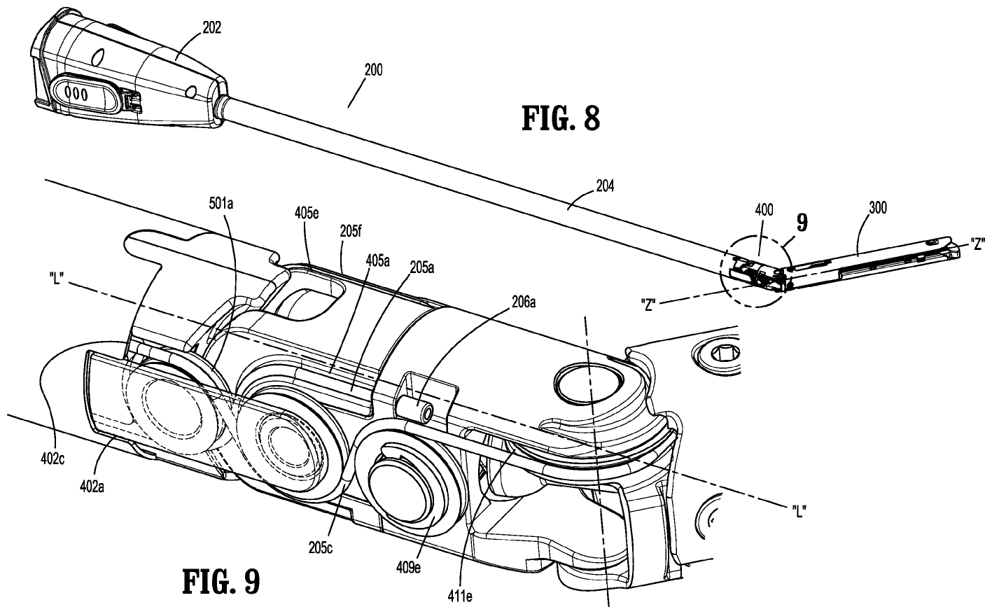

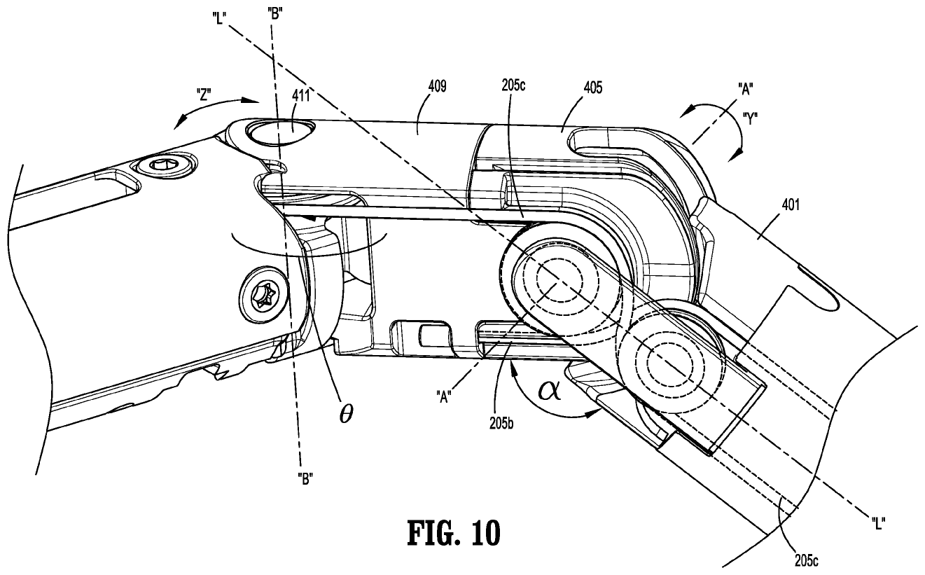

With reference to FIG. 3, end effector 300 of electromechanical surgical instrument 200 includes a mounting portion 302 on a proximal end portion thereof, and a first jaw member 304 (e.g., an anvil) and a second jaw member 306 (e.g., a cartridge assembly) that are coupled to mounting portion 302. First and second jaw members 304, 306 are positioned for pivotal movement between open (FIG. 3) and closed (FIG. 8) positions. First and second jaw members 304, 306 support a drive assembly 308 that is configured to fire a fastener cartridge 310 supported in second jaw member 306.

Mounting portion 302 defines a central opening (not shown) that is configured to receive drive coupler 238 of firing assembly 203b to couple drive coupler 238 to drive assembly 308 of end effector 300.

With reference to FIGS. 4 and 6, drive assembly 308 of end effector 300 includes a driven coupler 308a that is received in distal bore 238b of drive coupler 238 of firing assembly 203b of drive assembly 203. Driven coupler 308a of drive assembly 308 includes a non-circular configuration (e.g., D-shape) that is keyed to distal bore 238b of drive coupler 238 of firing assembly 203b so that driven coupler 308a and drive coupler 238 are rotatably locked with respect to one another such that driven coupler 308a and drive coupler 238 rotate together as drive coupler 238 rotates. Driven coupler 308a is pinned to a lead screw 308b (via pin 308p and pin 308pp) that supports a drive beam 308c such that rotation of driven coupler 308a causes lead screw 308b to rotate and axially advance drive beam 308c along lead screw 308b. For a more detailed description of components of example end effectors similar to end effector 300, reference can be made to U.S. Patent Application Publication Nos. 2016/0242779 and 2015/0297199, the entire disclosures of each of which are incorporated by reference herein.

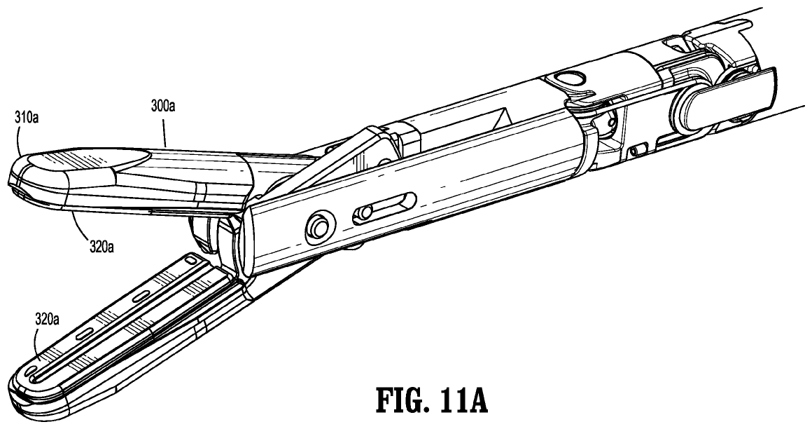

In use, with electromechanical surgical instrument 200 coupled to robotic surgical assembly 100 as seen in FIG. 1, one or more motors 50 of instrument drive unit 110 can be actuated to rotate one or more of driven members 209 of electrosurgical instrument 200 to push and/or pull one or more cables 205 of cable drive assembly 203a of drive assembly 203 of electromechanical surgical instrument 200. As cables 205 of cable drive assembly 203a axially translate, one or both of first link 405 (along with second link 409) and third link 411 of wrist assembly 400 rotate and/or articulate with one or more of proximal ball housing 406a, distal ball housing 406c, and/or dual ball shaft 234 of firing assembly 203b of drive assembly 203, relative to longitudinal axis “L-L.” In an aspect, each of first link 405 (along with second link 409) and third link 411 can be configured to articulate through an articulation angle of up to 90 degrees such that first link 405 (along with second link 409) can be articulated relative to first interface 401 through an articulation angle “a” up to 90 degrees and third link 411 can be articulated relative to second link 409 through an articulation angle “0” up to 90 degrees, as seen in FIG. 10. As can be appreciated, one or more components of firing assembly 203b pivot, rotate, and/or articulate as any of first interface 401, first link 405, second link 409, and third link 411 pivot, rotate, and/or articulate. In an aspect, a total range of motion is +/−55 degrees about axis “A-A” and +/−55 degrees about axis “B-B.”

While one or more components of firing assembly 203b pivot, rotate, and/or articulate as any of first interface 401, first link 405, second link 409, and third link 411 pivot, rotate, and/or articulate, firing assembly 203b can be rotated about longitudinal axis “L-L,” as indicated by arrow “D,” (see FIG. 4) in response to rotation of driven member 211 (FIG. 2B) by one or more of motors 50 of instrument drive unit 110 (FIG. 1). Rotation of firing assembly 203b of drive assembly 203 causes drive coupler 238 of firing assembly 203b to rotate lead screw 308b of end effector 300 about its axis, e.g., axis “Z-Z” (FIG. 8). Rotation of lead screw 308b of end effector 300 causes drive beam 308c of end effector 300 to advance distally along lead screw 308b, so that first and second jaw members 304, 306 of end effector 300 move from the open or unapproximated position (FIG. 3) thereof to the closed or approximated position (FIG. 8) thereof. As drive beam 308c of end effector 300 continues to advance distally along first and second jaw members 304, 306, drive beam 308c fires fastener cartridge 310 (FIG. 3) to fasten and/or sever tissue captured between first and second jaw members 304, 306 similar to that described in U.S. Patent Application Publication No. 2015/0297199 referenced above.

The effected articulation of the components of wrist assembly 400, as controlled by movement cables 205, will now be described in detail. As noted above, cable drive assembly 203a of electromechanical surgical instrument 200 includes one or more driven members 209, such as first driven member 209a, second driven member 209b, third driven member 209c, and fourth driven member 209d to enable robotic surgical assembly 100 to transfer power and actuation forces from motors 50 of robotic surgical assembly 100 to ultimately drive movement of components of end effector 300 (e.g., wrist assembly 400) of electromechanical surgical instrument 200. In particular, rotation of driven member 209a in a first direction (e.g., clockwise) effects proximal axial translation of first cable 205a, which in turn, causes first link 405 (and second link 409) to rotate relative to the first interface 401 about first pivot axis “A-A” in a first direction of arrow “Y” (FIG. 10). Rotation of driven member 209a in the first direction (e.g., clockwise) is met with simultaneous rotation of driven member 209b in a second, opposite, direction (e.g., counter-clockwise) which effects distal axial translation of second cable 205b. Similarly, rotation of driven member 209b in a first direction (e.g., clockwise) effects proximal axial translation of second cable 205b, which in turn, causes first link 405 (and second link 409) to rotate relative to first interface 401 about first pivot axis “A-A” in a second direction of arrow “Y” (FIG. 10). Rotation of driven member 209b in the first direction (e.g., clockwise) is met with simultaneous rotation of driven member 209a in a second, opposite, direction (e.g., counter-clockwise) which effects distal axial translation of first cable 205a.

Additionally, rotation of driven member 209c in a first direction (e.g., clockwise) effects proximal axial translation of one side of third cable 205c, which in turn, causes third link 411 to rotate relative to second link 409 about second pivot axis “B-B” in a first direction of arrow “Z” (FIG. 10). Rotation of driven member 209c in the first direction (e.g., clockwise) is met with simultaneous rotation of driven member 209d in a second, opposite, direction (e.g., counter-clockwise) which effects distal axial translation of the other side of third cable 205c. Similarly, rotation of driven member 209d in the first direction (e.g., clockwise) effects proximal axial translation of one side of third cable 205c, which in turn, causes third link 411 to rotate relative to second link 409 about second pivot axis “B-B” in a second direction of arrow “Z” (FIG. 10). Rotation of driven member 209d in the first direction (e.g., clockwise) is met with simultaneous rotation of driven member 209d in a second, opposite, direction (e.g., counter-clockwise) which effects distal axial translation of the other side of third cable 205c.

Although electromechanical surgical instrument 200 is described herein in connection with robotic surgical system 1, the presently disclosed electromechanical surgical instruments 200 can be provided in the form of a hand held electromechanical instrument, which may be manually driven and/or powered. For instance, U.S. Patent Application Publication No. 2015/0297199, referenced above, describes one example of a powered hand held electromechanical instrument, one or more of the components of which (e.g., the surgical device or handle thereof) can be utilized in connection with the presently disclosed surgical instrument 200.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

1. A robotic electromechanical surgical instrument, comprising:
a housing;
an elongated shaft defining a longitudinal axis and extending distally from the housing;
a wrist assembly supported on the elongated shaft and configured to articulate relative to the longitudinal axis, the wrist assembly including a first interface, a first link pivotally coupled to the first interface, a second link coupled to the first link, and a third link pivotally coupled to the second link;
a first cable coupled to the second link such that proximal axial translation of the first cable along the longitudinal axis causes the second link to pivot about a first pivot axis in a first direction;
a second cable coupled to the second link such that proximal axial translation of the second cable along the longitudinal axis causes the second link to pivot about the first pivot axis in a second direction opposite the first direction;
a third cable coupled to the third link such that axial translation of the third cable along the longitudinal axis causes the third link to pivot about a second pivot axis; and
an end effector coupled to the wrist assembly.

2. The robotic electromechanical surgical instrument of claim 1, wherein the first link and the second link are coupled together such that proximal axial translation of the first cable along the longitudinal axis causes the second link and the first link to pivot together about the first pivot axis.

3. The robotic electromechanical surgical instrument of claim 1, wherein the third cable is coupled to the third link such that proximal axial translation along the longitudinal axis of a first portion of the third cable, and simultaneous distal axial translation along the longitudinal axis of a second portion of the third cable, causes the third link to pivot about the second pivot axis in a third direction.

4. The robotic electromechanical surgical instrument of claim 3, wherein the third cable is coupled to the third link such that distal axial translation along the longitudinal axis of the first portion of the third cable, and simultaneous proximal axial translation along the longitudinal axis of the second portion of the third cable, causes the third link to pivot about the second pivot axis in a fourth direction opposite the third direction.

5. The robotic electromechanical surgical instrument of claim 1, further comprising an electrical cable operably coupled to a portion of at least one of the end effector or the wrist assembly.

6. The robotic electromechanical surgical instrument of claim 5, wherein the electrical cable is configured to transmit a sensor signal from at least one of the end effector or the wrist assembly.

7. The robotic electromechanical surgical instrument of claim 5, wherein the electrical cable is configured to transmit electrosurgical treatment energy to a portion of the end effector.

8. The robotic electromechanical surgical instrument of claim 5, wherein the housing includes an electrical contact disposed thereon and the electrical cable is coupled to the electrical contact.

9. The robotic electromechanical surgical instrument of claim 1, further comprising a firing assembly operably coupled to the end effector and configured to control an operation of the end effector.

10. The robotic electromechanical surgical instrument of claim 1, wherein the first interface includes a first half and a second half, wherein the first half defines a first cable channel slidably supporting the first cable therein and a third cable channel slidably supporting a first portion of the third cable therein and wherein the second half defines a second cable channel slidably supporting the second cable therein and a fourth cable channel slidably supporting a second portion of the third cable therein.

11. The robotic electromechanical surgical instrument of claim 10, wherein the second half of the first interface further defines an electrical cable channel configured to slidably support an electrical cable therein.

12. A wrist assembly for use with an electromechanical surgical instrument, the wrist assembly comprising:
a first interface defining a longitudinal axis;
a first link pivotally coupled to the first interface and configured to pivot relative to the first interface about a first pivot axis;
a second link coupled to the first link and axially aligned with the first link;
a third link pivotally coupled to the second link and configured to pivot relative to the second link about a second pivot axis;
a first cable coupled to the second link such that proximal axial translation of the first cable along the longitudinal axis causes the second link to pivot about the first pivot axis in a first direction;
a second cable coupled to the second link such that proximal axial translation of the second cable along the longitudinal axis causes the second link to pivot about the first pivot axis in a second direction opposite the first direction; and
a third cable coupled to the third link such that axial translation of the third cable along the longitudinal axis causes the third link to pivot about the second pivot axis.

13. The wrist assembly of claim 12, wherein the first link and the second link are coupled together such that proximal axial translation of the first cable along the longitudinal axis causes the second link and the first link to pivot together about the first pivot axis.

14. The wrist assembly of claim 12, wherein the third cable is coupled to the third link such that proximal axial translation along the longitudinal axis of a first portion of the third cable, and simultaneous distal axial translation along the longitudinal axis of a second portion of the third cable, causes the third link to pivot about the second pivot axis in a third direction.

15. The wrist assembly of claim 14, wherein the third cable is coupled to the third link such that distal axial translation along the longitudinal axis of the first portion of the third cable, and simultaneous proximal axial translation along the longitudinal axis of the second portion of the third cable, causes the third link to pivot about the second pivot axis in a fourth direction opposite the third direction.

16. The wrist assembly of claim 12, further comprising an electrical cable operably coupled to a portion of the wrist assembly.

17. The wrist assembly of claim 16, wherein the electrical cable is configured to transmit a sensor signal from the wrist assembly.

18. The wrist assembly of claim 12, wherein the first interface includes a first half and a second half, wherein the first half defines a first cable channel slidably supporting the first cable therein and a third cable channel slidably supporting a first portion of the third cable therein and wherein the second half defines a second cable channel slidably supporting the second cable therein and a fourth cable channel slidably supporting a second portion of the third cable therein.

19. The wrist assembly of claim 12, wherein the second half of the first interface further defines an electrical cable channel configured to slidably support an electrical cable therein.

20. The wrist assembly of claim 12, wherein the first interface defines a central channel along the longitudinal axis configured to support a portion of a drive assembly therethrough.
